(19) United States
(12) Patent Application Publication
(10) Pub. No.: US 2022/0101518 A1
(43) Pub. Date: Mar. 31, 2022

(54) SYSTEM AND METHOD FOR STYLIZING A MEDICAL IMAGE

(71) Applicant: GE Precision Healthcare LLC, Wauwatosa, WI (US)

(72) Inventor: Yelena Tsymbalenko, Mequon, WI (US)

(21) Appl. No.: 17/033,249

(22) Filed: Sep. 25, 2020

(51) Int. Cl. G06T 7/00; G06T 7/11; G16H 50/30; G16H 30/20

(52) U.S. Cl. CPC G06T 7/0012; G06T 7/11; G16H 50/30; G16H 30/20; G06T 2207/20084; G06T 2207/10024; G06T 2207/30084; G06T 2207/20081; G06T 2207/10132

(57) ABSTRACT

The present disclosure relates to stylizing a medical image. In accordance with certain embodiments, a method includes generating a medical image, segmenting the medical image into a first region and a second region, applying a first style to the first region and a different second style to the second region thereby generating a stylized medical image, and displaying the stylized medical image.

### TECHNICAL FIELD

This disclosure relates to a system and method for styling medical images and more particularly to system and method for styling ultrasound images.

### BACKGROUND

In order to visual internal structures, a clinician may order a patient undergoes various medical imaging procedures (i.e., a positron emission tomography (PET) scan, a computed tomography (CT) scan, a magnetic resonance imaging (MRI) procedure, an X-ray imaging procedure, etc.). Often, the medical images are displayed in a single color scheme (i.e., black and white) which may make it difficult for a physician to identify and follow-up on a health state/status of an anatomical structure(s)/organ(s) as the anatomical structure(s)/organ(s) may blend into the remainder of the image.

### SUMMARY

In one embodiment, the present disclosure provides a method. The method comprises generating a medical image, segmenting the medical image into a first region and a second region, applying a first style to the first region and a different second style to the second region thereby generating a stylized medical image, and displaying the stylized medical image.

In another embodiment, the present disclosure provides a system. The system comprises a processor and a computer readable storage medium that is in communication with the processor. When the processor executes program instructions stored in the computer readable storage medium, the processor receives a medical image, segments the medical image into a first region and a second region, applies a first style to the first region and a second style to the second region thereby generating a stylized medical image, and outputs the stylized medical image to a display.

In yet another embodiment, the present disclosure provides a computer readable storage medium with computer readable program instructions that, when executed by a processor, cause the processor to identify an anatomical structure within a medical image, segment the medical image into a first region and a second region, wherein the first region includes the anatomical structure, apply a first color scheme to the first region as a function of least one of a biomarker, a size of the anatomical structure, a disease state corresponding to the anatomical structure, an examination parameter relating to a patient, or a demographic relating to the patient, wherein the first color scheme is a monochromatic color scheme, apply a different second color scheme to the second region, thereby generating a stylized medical image, and output the stylized medial image to a display.

### BRIEF DESCRIPTION OF THE DRAWINGS

Various aspects of this disclosure may be better understood upon reading the following detailed description with reference to the drawings in which:

FIG. 1 is a schematic diagram of a medical imaging system in accordance with an exemplary embodiment;

FIG. 2 is a schematic diagram of an ultrasound system in accordance with an exemplary embodiment;

FIG. 3 is a schematic diagram of ultrasound components of an ultrasound system in accordance with an exemplary embodiment;

FIG. 4 is a schematic diagram of a cloud computing environment in accordance with an exemplary embodiment;

FIG. 5 is a flow chart of a method for stylizing a medical image in accordance with an exemplary embodiment;

FIG. 6 depicts a medical image in accordance with an exemplary embodiment;

FIG. 7 depicts a monochromatic color scheme in accordance with an exemplary embodiment; and

FIG. 8 depicts a stylized image in accordance with an exemplary embodiment;

FIG. 9 depicts another stylized image in accordance with an exemplary embodiment; and

FIG. 10 depicts a plurality of stylized images in accordance with an exemplary embodiment.

The drawings illustrate specific acts of the described components, systems, and methods for stylizing a medical image. Together with the following description, the drawings demonstrate and explain the structures, methods, and principles described herein. In the drawings, the thickness and size of components may be exaggerated or otherwise modified for clarity. Well-known structures, materials, or operations are not shown or described in detail to avoid obscuring aspects of the described components, systems, and methods.

### DETAILED DESCRIPTION

One or more specific embodiments of the present disclosure are described below. These described embodiments are only examples of the systems and methods for stylizing a medical image. The skilled artisan will understand that specific details described in the embodiments can be modified when being placed into practice without deviating from the spirit of the present disclosure.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (i.e., a material, element, structure, number, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

Referring to the figures generally, the present disclosure describes systems and methods for stylizing medical images. Medical images are often displayed in single color scheme (i.e., black and white) making it difficult to visualize a health state/status and follow-up changes related to anatomical structures and making it difficult to combine and visualize information regarding different anatomical structures/organs displayed in a same image.

Some embodiments of the present disclosure provide systems and methods that apply color to regions within a medical image. Applying color may make it easier to visualize health state of the anatomical structures/organs and make it easier to visualize combined information regarding one or more anatomical structures/organs displayed in a same image. Furthermore, some embodiments of the present disclosure provide systems and methods that colorize different segmented anatomical structures/organs across multiple patient visits which may aid in visualizing health changes for an anatomical structure/organ. Some embodiments apply the color according to a color scheme. Applying color according to a color scheme may convey a meaning (i.e., health of a region) that otherwise would not be visually conveyed. Other embodiments of the present disclosure provide systems and methods that apply an audible style (i.e., one or more musical notes, a message relating to the health of an anatomical structure, etc.) to a region in a medical image which may convey a meaning that is not visually conveyed.

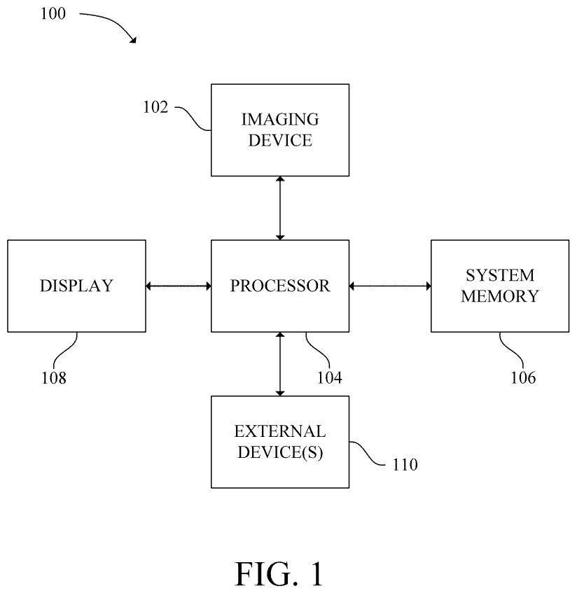

Referring now to FIG. 1, a medical imaging system 100 is shown in accordance with an exemplary embodiment. As illustrated in FIG. 1, in some embodiments, the medical imaging system 100 includes a medical imaging device 102, a processor 104, a system memory 106, a display 108, and one or more external devices 110.

The medical imaging device 102 may be any imaging device capable of capturing image data of a patient (i.e., PET, CT, MRI, X-ray machine, ultrasound imaging device etc.). Particularly, the medical imaging device 102 may be an ultrasound device. The medical imaging device 102 is in communication with the processor 104 via a wired or wireless connection thereby allowing the medical imaging device 102 to receive data from/send data to the processor 104. In one embodiment, the medical imaging device 102 may be connected to a network (i.e., a wide area network (WAN), a local area network (LAN), a public network (the Internet), etc.) which allows the medical imaging device 102 to transmit data to and/or receive data from the processor 104 when the processor 104 is connected to the same network. In another embodiment, the medical imaging device 102 is directly connected to the processor 104 thereby allowing the medical imaging device 102 to transmit data directly to and receive data directly from the processor 104.

The processor 104 may be a processor of a computer system. A computer system may be any device/system that is capable of processing and transmitting data (i.e., tablet, handheld computing device, smart phone, personal computer, laptop, network computer, etc.). In one embodiment, the processor 104 may include a central processing unit (CPU). In another embodiment, the processor 104 may include other electronic components capable of executing computer readable program instructions, such as a digital signal processor, a field-programmable gate array (FPGA), a graphics processing unit (GPU) or a graphics board. In yet another embodiment, the processor 104 may be configured as a graphical processing unit with parallel processing capabilities. In yet another embodiment, the processor 104 may include multiple electronic components capable of carrying out computer readable instructions. For example, the processor 104 may include two or more electronic components selected from a list of electronic components including: a CPU, a digital signal processor, an FPGA, GPU and a graphics board.

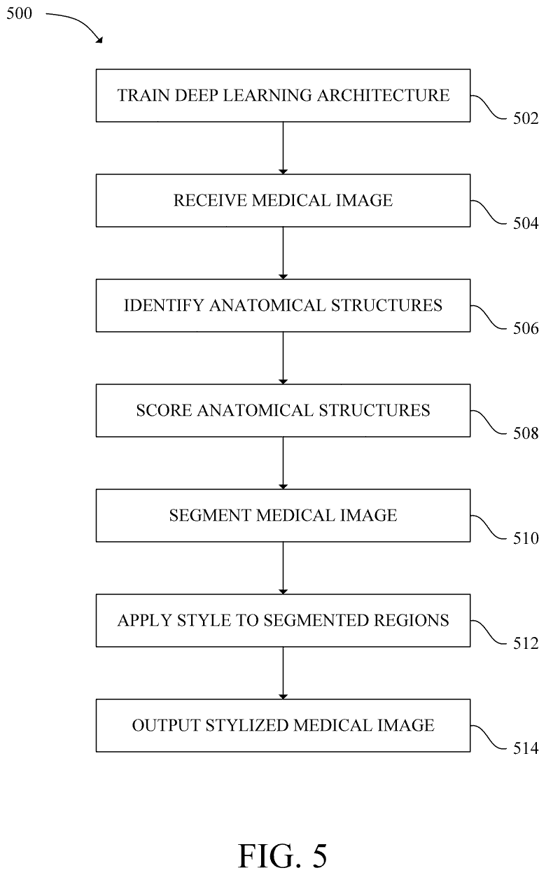

The processor 104 is in communication with the system memory 106. The system memory 106 is a computer readable storage medium. As used herein a computer readable storage medium is any device that stores computer readable instructions for execution by a processor and is not construed as being transitory per se. Computer readable program instructions include, but are not limited to, logic, data structures, modules, architecture, etc. that when executed by a processor create a means for implementing functions/acts specified in FIG. 5. Computer readable program instructions when stored in a computer readable storage medium and executed by a processor direct a computer system and/or another device to function in a particular manner such that a computer readable storage medium comprises an article of manufacture. System memory as used herein includes volatile memory (i.e., random access memory (RAM), and dynamic RAM (DRAM)) and nonvolatile memory (i.e., flash memory, read-only memory (ROM), magnetic computer storage devices, etc.). In some embodiments, the system memory may further include cache.

The display 108 and the one or more external devices 110 are connected to and in communication with the processor 104 via an input/output (I/O) interface. The one or more external devices 110 include devices that allow a user to interact with/operate the medical imaging device 102 and/or a computer system that includes the processor 104. As used herein, external devices include, but are not limited to, a mouse, keyboard, a touch screen, and a speaker.

The display 108 displays a graphical user (GUI). As used herein, a GUI includes editable data (i.e., patient data) and/or selectable icons. A user may use an external device to select an icon and/or edit the data. Selecting an icon causes a processor to execute computer readable program instructions stored in a computer readable storage medium which cause the processor to perform various tasks. For example, a user may use an external device 110 to select an icon which causes the processor 104 to control the medical device 102 to acquire image data of a patient.

When the processor 104 executes computer readable program instructions to begin image acquisition, the processor 104 sends a signal to begin imaging to the medical imaging device 102. In response, the medical imaging device 102 captures image data and sends the captured image data to the processor 104. In one example, the medical imaging device 102 may be a CT scanner. A CT scanner includes a radiation source, such as an X-ray tube, and a radiation sensitive detector opposite the radiation source. In response to receiving the signal to begin imaging, the radiation source emits radiation. The radiation traverses and is attenuated by a patient being imaged. The radiation sensitive detector detects the attenuated radiation and in response generates image data (i.e., projection image data). The radiation sensitive detector then sends the image data to the processor 104. According to other embodiments, different medical imaging systems may acquire ultrasound imaging data from an ultrasound device.

In response to receiving the image data, the processor 104 reconstructs the image data into one or more 2D digital imaging and communications in medicine (DICOM) images. In some embodiments, imaging may include moving the imaging device 102 while capturing image data. In this embodiment, the configured processor 104 may reconstruct the captured image data into a plurality of 2D images (or “slices”) of an anatomical structure. Furthermore, in some embodiments, the processor 104 may further execute computer readable program instructions to generate a 3D volume from the 2D slices.

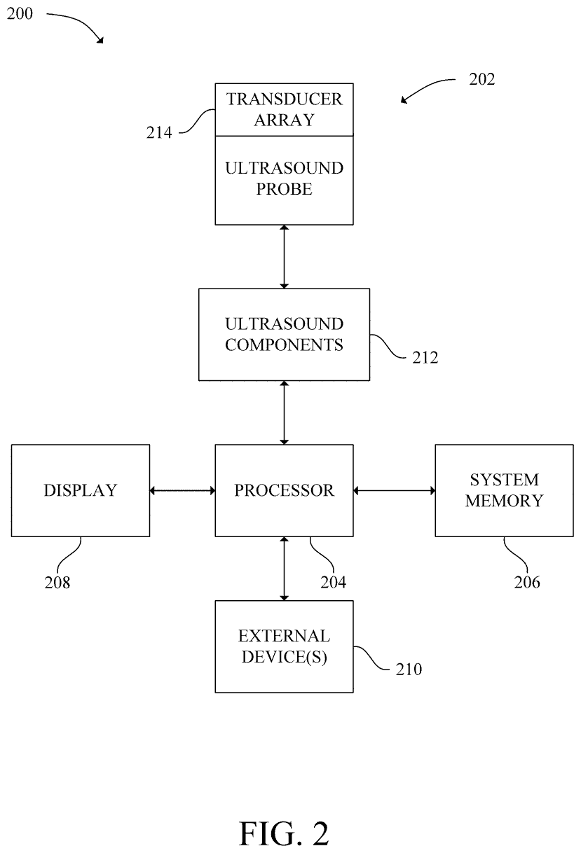

Referring now to FIG. 2, an ultrasound system 200 is shown in accordance with an exemplary embodiment. The ultrasound system 200 may serve as the medical imaging device 102. As shown in FIG. 2, in some embodiments, the ultrasound system 200 includes an ultrasound probe 202, a processor 204, a system memory 206, a display 208, one or more external devices 210, and ultrasound components 212.

The processor 204 may be a processor of a computer system. In one embodiment, the processor 204 may include a CPU. In another embodiment, the processor 204 may include other electronic components capable of executing computer readable program instructions. In yet another embodiment, the processor 204 may be configured as a graphical processing unit with parallel processing capabilities. In yet another embodiment, the processor may include multiple electronic components capable of carrying out computer readable program instructions. The processor 204 is in communication with the system memory 206. The system memory 206 is a computer readable storage medium.

The display 208 and the one or more external devices 210 are connected to and in communication with the processor 204 via an I/O interface. The one or more external devices 210 allow a user to interact with/operate the ultrasound probe 202 and/or a computer system with the processor 204.

The ultrasound probe 202 includes a transducer array 214. The transducer array 214 includes, in some embodiments, an array of elements that emit and capture ultrasonic signals. In one embodiment, the elements may be arranged in a single dimension (a “one-dimensional transducer array”). In another embodiment, the elements may be arranged in two dimensions (a two-dimensional transducer array”). Furthermore, the transducer array 214 may be a linear array of one or several elements, a curved array, a phased array, a linear phased array, a curved phased array, etc. The transducer array 214 may be a 1D array, a 1.25D array, a 1.5D array, a 1.75D array, or a 2D array according to various embodiments. Instead of an array of elements, other embodiments may have a single transducer element.

The transducer array 214 is in communication with the ultrasound components 212. The ultrasound components 212 connect the transducer array 214, and therefore the ultrasound probe 202, to the processor 204 via a wired or wireless connection. The processor 204 may execute computer readable program instructions stored in the system memory 206 which may cause the transducer array 214 to acquire ultrasound data, activate a subset of elements, and emit an ultrasonic beam in a particular shape.

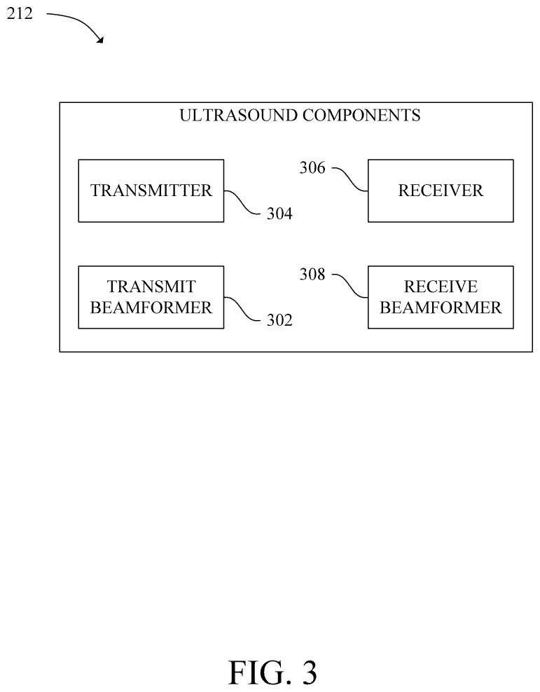

Referring now to FIG. 3, the ultrasound components 212 are shown in accordance with an exemplary embodiment. As shown in FIG. 3, in some embodiments, the ultrasound components 212 include a transmit beamformer 302, a transmitter 304, a receiver 306, and a receive beamformer 308. With reference to FIGS. 2 and 3, when the processor 204 executes computer readable program instructions to begin image acquisition, the processor 204 sends a signal to begin acquisition to the transmit beamformer 302. The transmit beamformer 302 processes the signal and sends a signal indicative of imaging parameters to the transmitter 304. In response, the transmitter 304 sends a signal to generate ultrasonic waves to the transducer array 214. Elements of the transducer array 214 then generate and output pulsed ultrasonic waves into the body of a patient. The pulsed ultrasonic waves reflect off of features within the body (i.e., blood cells, muscular tissue, etc.) thereby producing echoes that return to and are captured by the elements. The elements convert the captured echoes into electrical signals which are sent to the receiver 306. In response the receiver 306 sends signals indicative of the electrical signals to the receive beamformer 306 which process the signals into ultrasound image data. The receive beamformer 306 then sends the ultrasound data to the processor 204. The terms “scan” or scanning” may be used herein to refer to the processor of acquiring data through the process of transmitting and receiving ultrasonic signals. The ultrasound probe 202 may include all or part of the electronic circuitry to do all or part of the transmit and/or the receive beamforming. For example, all or part of the ultrasound components 212 may be situated within the ultrasound probe 202.

The processor 204 may further execute computer readable program instructions stored in the system memory 206 to further process the ultrasound data. In one embodiment, the processor 204 may process the ultrasound data into a plurality of 2D slices wherein each slice corresponds to a pulsed ultrasonic wave. In this embodiment, when the ultrasound probe 202 is moved during a scan, each slice may include a different segment of an anatomical structure. In some embodiments, the processor 204 may further process the slices to generate a 3D volume. The processor 204 may output a slice or a 3D volume to the display 208.

The processor 204 may further execute computer readable program instructions which cause the processor 204 to perform one or more processing operations on the ultrasound data according to a plurality of selectable ultrasound modalities. The ultrasound data may be processed in real-time during a scan as the echo signals are received. As used herein, the term “real-time” includes a procedure that is performed without any intentional delay. For example, the ultrasound probe 202 may acquire ultrasound data at a real-time rate of 7-20 volumes/second. The ultrasound probe 202 may acquire 2AD data of one or more planes at a faster rate. It is understood that real-time volume-rate is dependent on the length of time it takes to acquire a volume of data. Accordingly, when acquiring a large volume of data, the real-time volume-rate may be slower.

The ultrasound data may be temporarily stored in a buffer (not shown) during a scan and processed in less than real-time in a live or off-line operation. In one embodiment, wherein the processor 204 includes a first processor 204 and a second processor 204, the first processor 204 may execute computer readable program instructions that cause the first processor 204 to demodulate radio frequency (RF) data and the second processor 204, simultaneously, may execute computer readable program instructions that cause the second processor 204 to further process the ultrasound data prior to displaying an image.

The ultrasound probe 202 may continuously acquire data at, for example, a volume-rate of 21-30 hertz (Hz). Images generated from ultrasound data may be refreshed at a similar framerate. Other embodiments may acquire and display data at different rates (i.e., greater than 30 Hz or less than 10 Hz) depending on the size of the volume and intended application. In one embodiment, the system memory 206 stores at least several seconds of volumes of ultrasound data. The volumes are stored in a manner to facilitate retrieval thereof according to order or time of acquisition.

In various embodiments, the processor 204 may execute various computer readable program instructions to process the ultrasound data by other different mode-related modules (i.e., B-mode, Color Doppler, M-mode, Color M-mode, spectral Doppler, Elastography, TVI, strain, strain rate, etc.) to form 2D or 3D ultrasound data. Image lines and/or volumes are stored in the system memory 206 with timing information indicating at time at which the data was acquired. The modules may include, for example, a scan conversion mode to perform scan conversion operations to convert the image volumes from beam space coordinates to display space coordinates. A video processing module may read the image volumes stored in the system memory 206 and cause the processor 204 to generate and output an image to the display 208 in real-time whole a scan is being carried out.

While FIG. 2 depicts the processor 204, the system memory 206, the display 208, and the external devices 210 as separate from the ultrasound probe 202, in some embodiments, one or more of the processor 204, the system memory 206, the display 208, and the external devices 210 may be in the same device as the ultrasound probe 202. In various embodiments, the ultrasound probe 202 and the processor 204, the system memory 206, the display 208, and the external devices 210 may be in a separate handheld device.

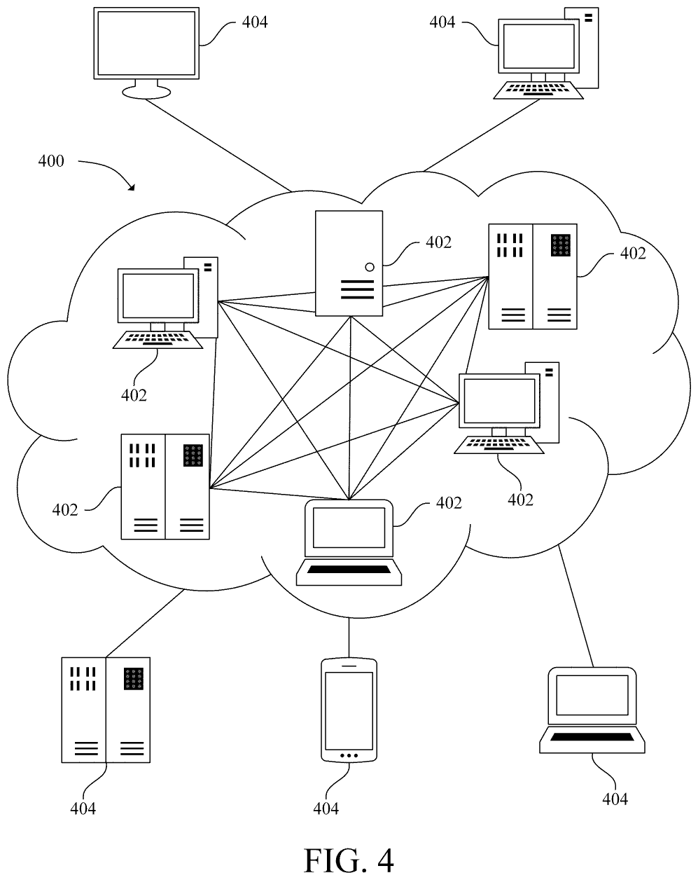

Referring now to FIG. 4, a cloud computing environment 400 is shown in accordance with an exemplary embodiment. As illustrated in FIG. 4, in some embodiments, the cloud computing environment 400 includes one or more nodes 402. Each node 402 may include a computer system/server (i.e., a personal computer system, a server computer system, a mainframe computer system, etc.). The nodes 402 may communicate with one another and may be grouped into one or more networks. Each node 402 may include a computer readable storage medium and a processor that executes instructions in the computer readable storage medium. As further illustrated in FIG. 4 one or more devices (or systems) 404 may be connected to the cloud computing environment 400. The one or more devices 404 may be connected to a same or different network (i.e., LAN, WAN, public network, etc.). The one or more devices 404 may include the medical imaging system 100 and the ultrasound system 200. One or more nodes 402 may communicate with the devices 404 thereby allowing the nodes 402 to provide software services to the devices 404.

In some embodiments the processor 104 or the processor 204 may output a generated image to a computer readable storage medium of a picture archiving and communication system (PACS). A PACS stores images generated by medical imaging devices and allows a user of a computer system to access the medical images. The computer readable storage medium that includes the PACS may be in a node 402 and/or another device 404. In some embodiments, the PACS is coupled to a remote system, such as a radiology department system, hospital information system, etc. A remote system allows operates at different locations to access the image data.

A processor of a node 402 or another device 404 may execute computer readable instructions in order to train a deep learning architecture. A deep learning architecture applies a set of algorithms to model high-level abstractions in data using multiple processing layers. Deep learning training includes training the deep learning architecture to identify features within an image (i.e., DICOM images) based on similar features in a plurality of training images that comprise a training data set. “Supervised learning” is a deep learning training method in which the training data set includes only images with already classified data. That is, the training data set includes images wherein a clinician has previously identified anatomical structures or regions of interest (i.e., organs, blood vessels, tumors, lesions, etc.) within each training image. “Semi-supervised learning” is a deep learning training method in which the training data set includes some images with already classified data and some images without classified data. “Unsupervised learning” is a deep learning training method in which the training data set includes only images without classified data but identifies abnormalities within the training data set. “Transfer learning” is a deep learning training method in which information stored in a computer readable storage medium that was used to solve a first problem is used to solve a second problem of a same or similar nature as the first problem (i.e., identify structures or regions of interest in a DICOM image).

Deep learning operates on the understanding that datasets include high level features which include low level features. While examining an image, for example, rather than looking for an object (i.e., organs, blood vessels, tumors, lesions, etc.) within an image, a deep learning architecture looks for edges which form parts, which form the an object being sought based on learned observable features. Learned observable features include objects and quantifiable regularities learned by the deep learning architecture during training. A deep learning architecture provided with a large training set of well classified data is better equipped to distinguish and extract features pertinent to successful classification of new data.

A deep learning architecture that utilizes transfer learning may properly connect data features to certain classifications affirmed by a human expert. Conversely, the same deep learning architecture can, when informed of an incorrect classification by a human expert, update the parameters for classification. Settings and/or other configuration information, for example, can be guided by learned se of settings and/or other configuration information, and as a system is used more (i.e., repeatedly and/or by multiple users), a number of variations and/or other possibilities for settings and/or other configuration information can be reduced for a given situation. Deep learning architecture can be trained on a set of expert classified data. This set of data builds the first parameters for the architecture and is the stage for supervised learning.

During supervised learning, the deep learning architecture can be tested to determine if a desired behavior has been achieved (i.e., the deep learning architecture has been trained to operate according to a specified threshold, etc.). Once a desired behavior has been achieved, the architecture can be deployed for use. That is, the deep learning architecture can be tested with “real” data. During operation, classifications made by the deep learning architecture can be confirmed or denied by an expert user, an expert system, or reference databases to continue to improve architecture behavior. The architecture is then in a state of transfer learning, as parameters for classification that determine architecture behavior are updated based on ongoing interactions. In certain examples, the architecture can provide direct feedback to another process. In certain examples, the architecture outputs data that is buffered (i.e., via the cloud computing environment 400) and validated before it is provided to another process.

Deep learning architecture can be applied via a computer assistance detection (CAD) system to analyze DICOM images that are generated by the medical imaging system 100, the ultrasound system 200, or stored in a PACS. Particularly, the deep learning architecture can be used to analyze 2D (and/or 3D) DICOM images to identify anatomical structures (i.e., organs, tumors, blood vessels, lesions, etc.) within a 2D and/or 3D image.

Referring now to FIG. 5, a flow chart of a method 500 for stylizing a medical image is shown in accordance with an exemplary embodiment. Various aspects of the method 500 may be carried out by a “configured processor.” As used herein a configured processor is a processor that is configured according to an aspect of the present disclosure. A configured processor(s) may be the processor 104 or the processor 204. A configured processor executes various computer readable computer readable program instructions to perform the steps of the method 500. The computer readable program instructions, that when executed by a configured processor, cause a configured processor to carry out the steps of the method 500 may be stored in the system memory 106, the system memory 206, system memory of a node 402 or a system memory of another device 404. The technical effect of the method 500 is stylizing a medical image.

At 502, a configured processor trains a deep learning architecture with a plurality of 2D images (“the training dataset”). The plurality of 2D images include, but are not limited to, images generated by a CT system, a PET system, an MM system, an X-ray system, and an ultrasound system. The plurality of 2D images may include DICOM images. The deep learning architecture is trained via supervised, semi-supervised, unsupervised and transfer learning as previously described herein to identify anatomical structures within individual training images. After training, the configured processor applies the deep learning architecture to a test dataset of 2D images. The deep learning architecture identifies anatomical structures within individual images of the test dataset. In some embodiments, the configured processor then checks the accuracy of the deep learning architecture by comparing the anatomical structures identified by the deep learning architecture to a ground truth mask. As used herein, a ground truth mask is a mask that includes accurately identified anatomical structures. In other embodiments, a clinician checks the accuracy of the deep learning architecture. If the deep learning architecture does not achieve a threshold level of accuracy (i.e., 80% accuracy, 90% accuracy, 95% accuracy, etc.) in identifying anatomical structures, then the configured processor continues to train the deep learning architecture until the desired accuracy is achieved. When the desired accuracy is achieved, the deep learning architecture can be applied to datasets with images that do not include previously identified anatomical structures.

At 504, the configured processor receives a 2D DICOM image from the imaging system 100, the ultrasound system 200, or a PACS.

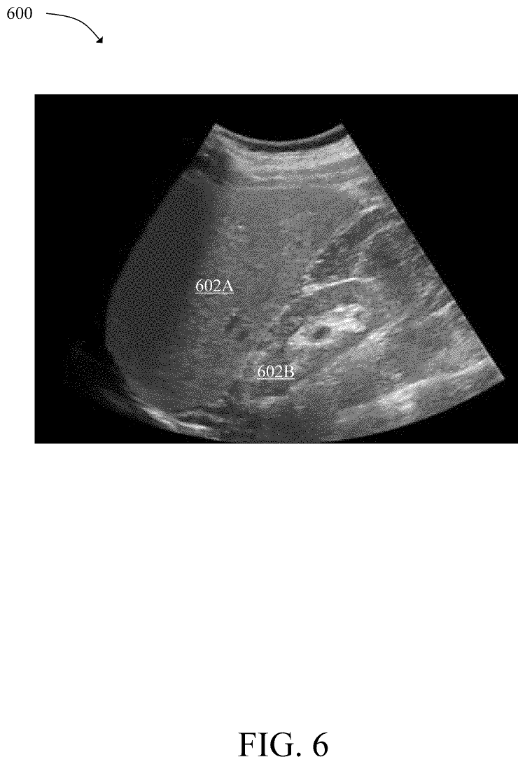

At 506, the configured processor identifies at least one anatomical structure (i.e., a “first anatomical structure,” a “second anatomical structure, a “third anatomical structure,” etc.) within the 2D DICOM image with the deep learning architecture. The anatomical structures may include, but are not limited to organs, blood vessels, tumors, and lesions. In one example, the configured processor identifies one anatomical structure (a “first anatomical structure”) with the deep learning architecture. In another example, the configured processor identifies two anatomical structures (a “first anatomical structure” and a “second anatomical structure”). Briefly turning to FIG. 6, a 2D DICOM image 600 is shown in accordance with an exemplary embodiment. In this embodiment, the 2D DICOM image is produced from ultrasound data. The 2D DICOM image 600 includes a first anatomical structure 602A and a second anatomical structure 602B. In this example, the first anatomical structure 602A and the second anatomical structure 602B are different organs. Specifically, the first anatomical structure 602A corresponds to the liver and the second anatomical structure 602B corresponds to a kidney.

At 508, the configured processor scores the identified anatomical structures as a function of a health (i.e., a health state or status) identified anatomical structure. The configured processor determines the health of the identified anatomical structure as a function of biomarkers, a size of the identified anatomical structure, a disease state corresponding to the identified anatomical structure, examination parameters relating to the patient (i.e., body mass index (BMI), weight, blood pressure, resting heart rate, etc.), and demographics relating to the patient (i.e., age, ethnicity, gender, etc.). In some embodiments, the biomarkers correspond to an identified anatomical structure and/or a disease state that relates to an identified anatomical structure. In one example, wherein an identified anatomical structure is the liver, the biomarkers may include, but are not limited to, aspartate transaminase (AST), alanine transaminase (ALT), alkaline phosphatase (ALP), cholesterol, low-density lipoprotein (LDL), high-density lipoprotein (HDL), bilirubin, prothrombin time (PT), partial prothrombin time (PPT), albumin total protein, gamma-glutamyltransferase (GGT), L-lactate dehydrogenase (LD), and international normalized ratio. In another example wherein an identified anatomical structure is a kidney, the biomarker may include, but are not limited to blood urea nitrogen (BUN), glomerular filtration rate (GFR), neutrophil gelatinase-associated lipocalin (NGAL), kidney injury molecule-1 (KIM-1), and liver-type fatty acid binding protein (L-FABP). In yet another example, wherein an identified anatomical structure is a tumor, the biomarkers, may include but are not limited to alpha-fetoprotein (AFP), beta-2-microglobulin (B2M), beta-human chorionic gonadotropin (beta-hCG), fibrin/fibrinogen, lactate dehydrogenase, neuron-specific enolase (NSE), nuclear matrix protein 22, prostatic acid phosphatase (PAP), and thyroglobulin. In some embodiments, the configured processor automatically scores the identified anatomical structure and automatically determines the size of the anatomical structure including, but not limited to, a length of a long axis of the anatomical structure and a length of a short axis of the anatomical structure.

In some embodiments, configured processor assigns a higher score to a healthier anatomical structure and scores the anatomical structure on a scale of 1-10. In one example, a clinician may diagnose John Doe with non-alcoholic fatty liver disease (NAFLD). At a first examination a clinician diagnoses John Doe with stage 1 NAFLD. In this example, the configured processor may assign a score of 7 to the anatomical structure (the liver) in a first 2D DICOM image taken during the first examination as the liver is in the early stages of NAFLD. At a second examination, the clinician may diagnose John Doe with stage 3 NAFLD. In this example, the configured processor may assign a score of 4 to the anatomical structure (the liver) in a second 2D DICOM image taken during the second examination as the liver is in the later stages of NAFLD. The configured processor assigned a lower score at the second examination as the disease state corresponding to the anatomical structure has progressed.

In another example, a clinician may diagnose Jane Doe with breast cancer. At a first examination, a clinician may determine the tumor is 6 cm large. In this example, the configured processor may assign a score of 3 to the anatomical structure (the tumor) in a first 2D DICOM image taken during the first examination as the tumor is a T3 grade tumor. At a second examination, a clinician may determine the tumor is 1 cm large. In this example, the configured processor may assign a score of 7 to the anatomical structure (the tumor) in a second 2D DICOM image taken during the second examination as the tumor is a T1 grade tumor. The configured processor assigns a higher score at the second examination as the anatomical structure is smaller, which corresponds to a lower tumor grade.

In yet another example, wherein a clinician diagnosed Jane Doe with breast cancer, at a first examination the clinician may determine the tumor is 1 cm large. In this example, the configured processor may assign a score of 7 to the anatomical structure (the tumor) in a first 2D DICOM image taken during the first examination as the tumor is a T1 grade tumor. At a second examination, the clinician may determine the tumor is 6 cm large. The configured processor may assign a score of 3 to the anatomical structure (the tumor) in a second 2D DICOM image taken during the second examination as the tumor is a T3 grade tumor. The configured processor assigns a lower score at the second examination as the anatomical structure is larger, which corresponds to a higher tumor grade.

At 510, the configured processor segments the 2D DICOM image into at least two regions (i.e., a “a first region,” a “second region,” etc.) wherein at least one of the regions includes an identified anatomical structure. In some embodiments, the region that includes the identified anatomical structure includes only the identified anatomical structure. The configured processor may segment the 2D DICOM image according to a number of techniques. In one example, wherein the configured processor identified one anatomical structure at 506, the configured processor segments the 2D DICOM image into a first region and a second region, wherein the first region includes the anatomical structure and the second region does not include the anatomical structure. In another example, wherein the configured processor identified a first anatomical structure and a second anatomical structure at 506, the configured processor segments the 2D DICOM image into a first region that includes the first anatomical structure, a second region that includes the second anatomical structure, and a third region that does not include the first or second anatomical structures.

At 512, the configured processor applies a style to the segmented regions thereby generating a stylized 2D DICOM image. Applying a style to the segmented regions includes applying a style to individual pixels of the 2D DICOM image. As used herein, a style includes a color palette style, an audible style, and an imaging device style. In some embodiments, wherein the configured processor applies two styles (i.e., a first style and a second style) the first style and second styles are different and the configured processor automatically applies the styles.

A color palette style includes color schemes based on color wheel theory. Color schemes based on color wheel theory include, but are not limited to, monochromatic color schemes, temperature color schemes, complementary color schemes, analogous color schemes, triadic color schemes, split-complementary color schemes, tetradic color schemes, and square color schemes.

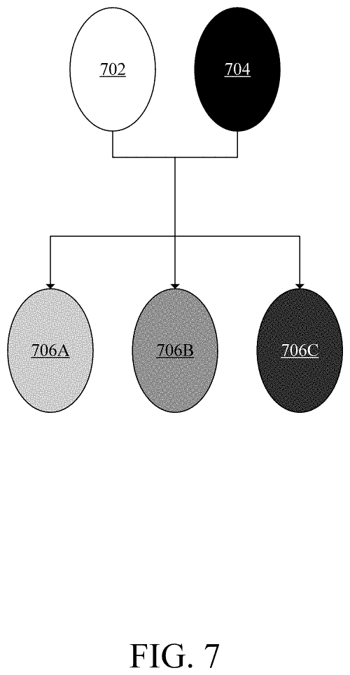

A monochromatic color scheme uses one hue and adds white, black, or gray, to tint, tone, and shade the hue. Briefly referring to FIG. 7, a monochromatic color scheme is shown in accordance with an exemplary embodiment. In this example, the monochromatic color scheme includes a white hue 702 and adds a varying amounts of a black tint 704 to create a first shade 706A, a second shade 706B, and a third shade 706C. A monochromatic color scheme may be used to illustrate the health of the anatomical structure. In one example, wherein the configured processor assigns a higher score to a healthier anatomical structure, when the configured processor assigns a low score (i.e., 2) to the anatomical structure the configured processor may apply a dark tint of a chosen hue to the anatomical structure as a dark tint may visually indicate the anatomical structure is in poor health. In another example, wherein the configured processor assigns a higher score to a healthier anatomical structure, when the configured processor assigns a high score (i.e., 9) to the anatomical structure the configured processor may applies a light tint of a chosen hue to the anatomical structure as a light tint may visually indicate the anatomical structure is in good health.

A temperature color scheme includes warm colors (i.e., reds, oranges, or yellows) and cool colors (i.e., purples, blues or greens). In some embodiments, the configured processor may apply a warm or cool color to the region as a function of an examination type. In one example, John Doe may undergo a routine medical imaging procedure. In this example, the configured processor may apply cool colors to an anatomical structure of a 2D DICOM image generated during the imaging procedure as cool colors may be associated with normal circumstances. In another example, John Doe may undergo a medical imaging procedure to determine the progression of a cancer. In this example, the configured processor may apply a warm color to an anatomical structure (i.e., a tumor) of a 2D DICOM image generated during the imaging procedure as warm colors may be associated with circumstances relating to a threat.

A complementary color scheme includes pairing opposite colors. Opposite colors (i.e., colors that sit across from each other on the color wheel) cancel each other out when combined. Complementary colors include, but are not limited to, red and green, purple and yellow, and orange and blue. In some embodiments the configured processor may apply complementary colors to the first and second regions to contrast the first region from the second region. In one example, a 2D DICOM image may include a first region that includes the liver and a second region that includes the kidney. In this example, the configured processor may apply a blue color to the first region and an orange color to the second region to contrast the kidney from the liver.

An analogous color scheme includes grouping 2-4 colors that are adjacent to one another on the color wheel. Analogous colors include, but are not limited to, red, orange and yellow, and purple, blue and green. In some embodiments, the configured processor may apply one color from a first group of analogous colors to the first region and another color from a different second group of analogous colors to the second region to contrast the first region from the second region.

A triadic color scheme includes grouping three colors that are evenly spaced around the color wheel. Triadic colors include, but are not limited to, orange, purple, and blue, and red, yellow, and a dark blue. The configured processor may deploy a triadic color scheme when the configured processor segments the 2D DICOM image into three regions. In some embodiments, wherein the configured processor segments a 2D DICOM image into a first region, a second region, and a third region, the configured processor may apply a yellow color to the first region, a red color to the second region, and a dark blue color to the third region to contrast the first, second, and third regions in a balanced manner.

A split-complementary color scheme includes grouping three colors as a function of a base color. The configured processor selects a base color and two colors adjacent to a color that is complementary to the base color. The configured processor may deploy a split-complementary color scheme when the configured processor segments the 2D DICOM image into three regions. In some embodiments, wherein the configured processor segments a 2D DICOM image into a first region, a second region, and a third region, the configured processor may assign the first region the base color, assign the second region a first color that is adjacent to a color that is complementary to the base color, and assign the third region a second color that is adjacent to a color that is complementary to the base color.

A tetradic color scheme includes grouping two pairs of complementary colors. A tetradic color scheme may include, but is not limited to, red, green, purple, and yellow. The configured processor may deploy a tetradic color scheme when the configured processor segments the 2D DICOM image into four regions. In some embodiments, wherein the configured processor segment a 2D DICOM image into a first region, a second region, a third region, and a fourth region, the configured processor may assign a red color to the first region, a green color to the second region, a purple color to the third region, and a yellow color to the fourth region to contrast the four regions.

A square color scheme includes grouping four colors that are evenly spaced around the color wheel. A square color scheme may include, but is not limited to, red, orange, purple, and green. The configured processor may deploy a square color scheme when the configured processor segments the 2D DICOM image into four regions. In some embodiments, wherein the configured processor segment a 2D DICOM image into a first region, a second region, a third region, and a fourth region, the configured processor may assign a red color to the first region, a purple color to the second region, a green color to the third region, and an orange color to the fourth region to contrast the four regions.

An audible style may include one or more musical notes, tones, rising or falling pitches, songs, etc. in same or changing volumes. The configured processor may assign different audible styles to different regions. In one example, wherein the configured processor segments a 2D DICOM image into a first region and a second region, the configured processor may assign a C note to the first region and an A note to the second region. In another example, wherein the configured processor segments a 2D DICOM image into a first region, a second region, and third region, the configured processor may assign a C note to the first region an F note to the second region, and an A note to the third region. An audible style may further include a message regarding a health state or a disease state of an anatomical structure. An audible style

An imaging device style may include one or more display styles relating to a medical imaging device (i.e., CT, MRI, ultrasound, X-ray, etc.) or a manufacture of a medical imaging device. For example, the configured processor may apply a CT image style to a 2D DICOM image/or segmented area(s) of a 2D DICOM image generated by an ultrasound system thereby making the 2D DICOM image appear as though a CT imaging system generated the 2D DICOM image. In another example, the configured processor may apply a style corresponding to medical imaging system of a first manufacture to a 2D DICOM image generated by a medical imaging system of a different second manufacturer.

At 514, the configured processor outputs the stylized image to the display 108 or the display 208. When the stylized image includes an audible style, when a user selects a region with an audible style with an external device 110 or an external device 210, the selection causes the processor to output the audible style to a speaker. In one example wherein the external device 110 or the external device 210 includes a touch screen of the display 108 or the display 208 and the configured processor outputs a stylized image with an audible style, a user touching a region that includes the audible style causes the configured processor to output the audible style to a speaker. In another example, wherein the external device 110 or the external device 210 includes a mouse and the configured processor outputs a stylized image with an audible style, a user clicking a region that includes the audible style causes the configured processor to output the audible style to a speaker. In some embodiments, the configured processor may save the stylized image to a system memory of a node 402, another device 404, or a system memory of a PACS.

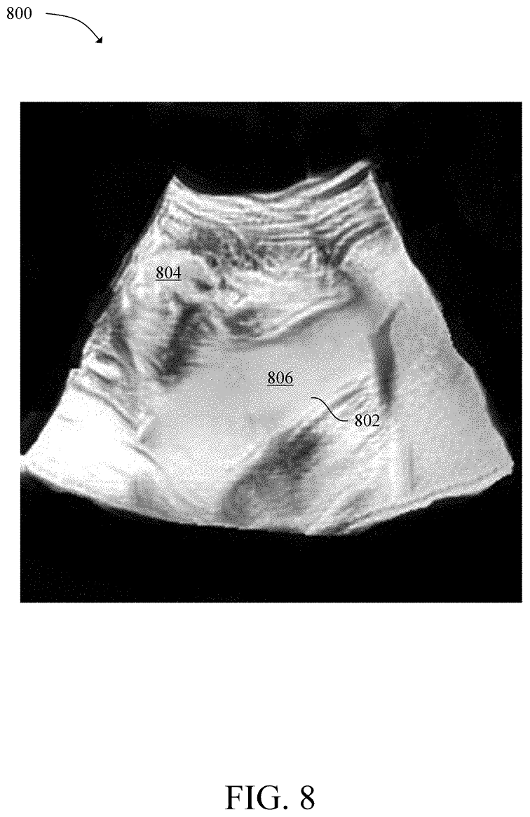

Referring now to FIG. 8, a first stylized image 800 is shown in accordance with an exemplary embodiment. In this embodiment, the 2D DICOM image which serves as the basis for the stylized image 800 is generated from ultrasound image data. The first stylized image 800 includes a first region 802 and a second region 804. The first region 802 includes a first anatomical structure 806. The first anatomical structure 806 includes a kidney of a patient being imaged. In this example, the configured processor applied a monochromatic color scheme and assigned a color according to the monochromatic color scheme as a function of determined health of the kidney. In this example, the configured processor may have scored the kidney with a score corresponding to good health (i.e., a score of 9 on a scale of 1-10 wherein 10 is a healthy kidney) and accordingly, assigned a lighter color to the first region 802 thereby depicting the kidney is in good health.

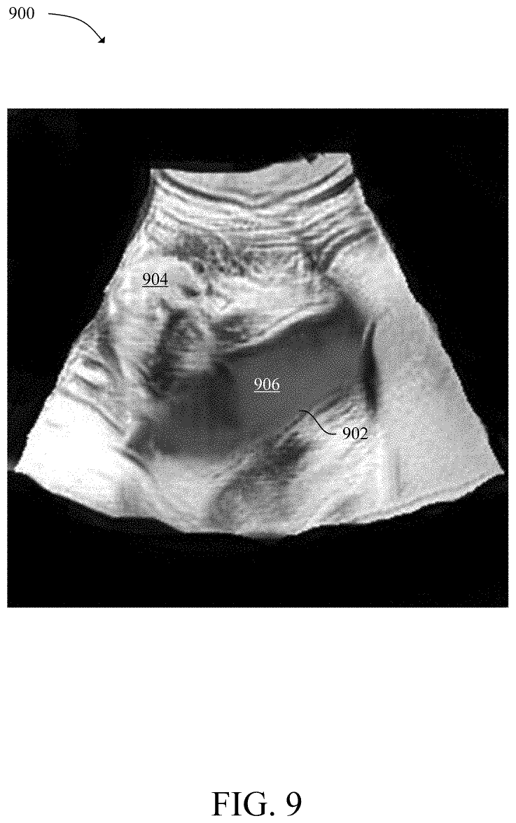

Referring now to FIG. 9, a second stylized image is shown in accordance with an exemplary embodiment. In this embodiment, the 2D DICOM image which serves as the basis for the stylized image 900 is generated from ultrasound image data. The second stylized image 900 includes a first region 902 and a second region 904. The first region 902 includes a first anatomical structure 906. The first anatomical structure 906 includes a kidney of a patient being imaged. In this example, the configured processor applied a monochromatic color scheme and assigned a color according to the monochromatic color scheme as a function of determined health of the kidney. In this example, the configured processor may have scored the kidney with a score corresponding to poor health (i.e., a score of 2 on a scale of 1-10 wherein 10 is a healthy kidney) and accordingly, assigned a darker color to the first region 902 thereby depicting the kidney is in poor health.

The steps of the method 500 may be applied to multiple 2D (or 3D) DICOM images across a number of patient visits. The configured processor may output stylized images from generated from 2D (or 3D) DICOM images taken across multiple patient visits individually or collectively as previously discussed herein. When stylized images from different patient visits are stored in a system memory, the configured processor may retrieve the stylized images from the system memory and output the stylized images as previously discussed herein.

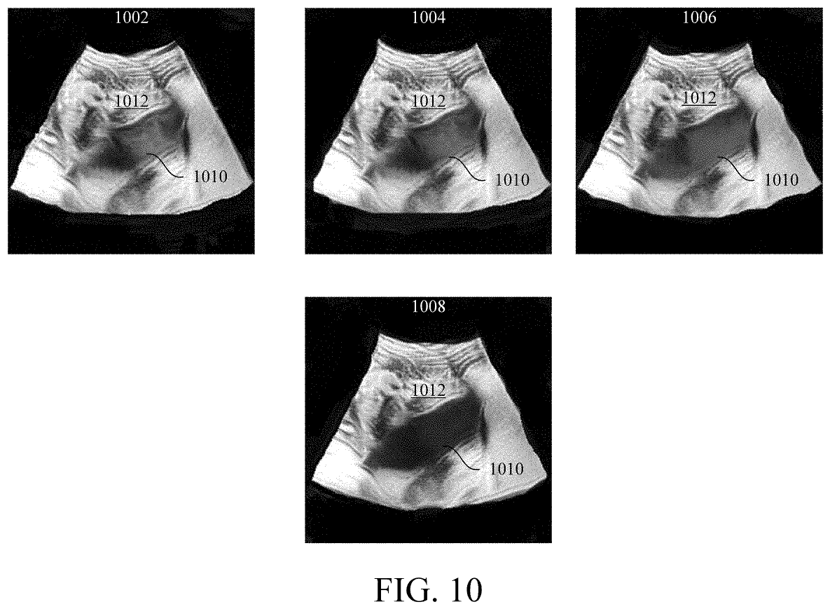

Outputting stylized images across multiple patient visits may aid a clinician in visualizing the progression of a disease state of an organ. For example, as depicted in FIG. 10, a configured processor may carry out the method 500 to generate and output a first stylized image 1002 from a first 2D (or 3D) DICOM image generated at a first patient visit, a second stylized image 1004 from a second 2D (or 3D) DICOM image generated at a second patient visit, a third stylized image 1006 from a third 2D (or 3D) DICOM image generated at a third patient visit, and a fourth stylized image 1008 from a fourth 2D (or 3D) DICOM image generated at a fourth patient visit. In this example, the configured processor identified a first region 1010 and a second region 1012 in each stylized image 1002-1008. The first region 1010 includes the kidney and the second region 1012 includes the remainder of the stylized images 1002-1008. In this example, the configured processor scored the health of the kidney on a scale of 1-10 as a function of a disease state of the kidney (i.e., chronic kidney disease (CKD)) at each patient visit wherein a score of 10 corresponds to a healthy kidney. At the first patient visit, the kidney was at stage 1 CKD, at the second patient visit the kidney was at stage 3 CKD, at the third patient visit the kidney was at stage 4 CKD, and at the fourth patient visit the kidney was at stage 5 CKD. Accordingly, the configured processor may score the kidney a 6 at the first patient visit, a 4 at the second patient visit, a 2 at the third patient visit, and a 1 at the fifth patient visit.

In this example, configured processor segmented a 2D DICOM image corresponding to each stylized image 1002-1008 into a first region 1010 and a second region 1012 and applied a monochromatic color scheme to the first region 1010 (the kidney) and a different color scheme to the second region 1012. Furthermore, the configured processor applied the color scheme to the first region 1010 as a function of the determined health, and therefore the determined score, of the kidney. As see in FIG. 10, the configured processor applied a darker hue to the first region 1010 as the health of the kidney deteriorated. This visual progression of a darkening color may aid a clinician or patient in visualizing the health of the kidney. Furthermore, a darkening color may convey that the health of the kidney is deteriorating as darker colors may be associated with harmful circumstances. While the above example describes applying a color scheme to one anatomical structure in a 2D (or 3D)DICOM, it is understood that the above method could be applied to more than one anatomical structure which allows a clinician to independently visualize a health state or disease progression of multiple anatomical structures within an image.

In addition to any previously indicated modification, numerous other variations and alternative arrangements may be devised by those skilled in the art without departing from the spirt and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments are meant to be illustrative only and should not be construed to be limiting in any manner.

What is claimed is:

1. A method comprising:
generating a medical image;
segmenting the medical image into a first region and a second region;
applying a first style to the first region and a different second style to the second region thereby generating a stylized medical image; and
displaying the stylized medical image.

2. The method of claim 1, further comprising:
generating the medical image from ultrasound image data.

3. The method of claim 1, further comprising:
identifying an anatomical structure in the medical image,
wherein the first region includes the anatomical structure and the second region includes a remainder of the image.

4. The method of claim 3, further comprising:
determining a health status of the anatomical structure; and
applying the first color style to the first region as a function of the determined health status of the anatomical structure.

5. The method of claim 4, wherein the health status of the anatomical structure is determined as a function of at least one of a biomarker, a size of the anatomical structure, a disease state corresponding to the anatomical structure, an examination parameter relating to a patient, or a demographic relating to the patient.

6. The method of claim 1, further comprising:
identifying a first anatomical structure and a different second anatomical structure in the medical image,
wherein the first region includes the first anatomical structure and the second region includes the second anatomical structure.

7. The method of claim 1, wherein the first and second style are one of a color palette style, an audible style, and an imaging device style.

8. The method of claim 7, wherein the first or second style is a color palette style selected from one of a monochromatic color scheme, a temperature color scheme, a complementary color scheme, an analogous color scheme, a triadic color scheme, a split-complementary color scheme, a tetradic color scheme, and a square color scheme.

9. A system comprising:
a processor;
a computer readable storage medium in communication with the processor, wherein the processor executes program instructions stored in the computer readable storage medium which cause the processor to:
receive a medical image;
segment the medical image into a first region and a second region;
apply a first style to the first region and a second style to the second region thereby generating a stylized medical image; and
output the stylized medical image to a display.

10. The system of claim 9, wherein the medical image is generated from ultrasound image data.

11. The system of claim 9, wherein the program instructions further cause the processor to:
identify an anatomical structure in the medical image, wherein the first region includes the anatomical structure.

12. The system of claim 11, wherein the instructions further cause the processor to:
determine a health status of the anatomical structure; and
apply the first color style to the first region as a function of the determined health status of the anatomical structure.

13. The system of claim 12, wherein the instructions further cause the processor to:
determine the health status of the anatomical structure as a function of a biomarker, a size of the anatomical structure, a disease state corresponding to the anatomical structure, an examination parameter relating to a patient, or a demographic relating to the patient.

14. The system of claim 9, wherein the program instructions further cause the processor to:
identify a first anatomical structure and a different second anatomical structure in the medical image,
wherein the first region includes the first anatomical structure and the second region includes the second anatomical structure.

15. The system of claim 9, wherein the first and second style are one of color palette style, an audible style, and an imaging device style.

16. The system of claim 15, wherein the first or second style is a color palette style selected from one of a monochromatic color scheme, a temperature color scheme, a complementary color scheme, an analogous color scheme, a triadic color scheme, a split-complementary color scheme, a tetradic color scheme, and a square color scheme.

17. The system of claim 16, wherein the instructions further cause the processor to:
apply the first or second style to the first or second region by applying a color of the color style palette to pixels of the first or second region.

18. A computer readable storage medium with computer readable program instructions that, when executed by a processor, cause the processor to:
identify an anatomical structure within a medical image;
segment the medical image into a first region and a second region, wherein the first region includes the anatomical structure;
apply a first color scheme to the first region as a function of at least one of a biomarker, a size of the anatomical structure, a disease state corresponding to the anatomical structure, an examination parameter relating to a patient, or a demographic relating to the patient, wherein the first color scheme is a monochromatic color scheme;
apply a different second color scheme to the second region, thereby generating a stylized medical image; and
output the stylized medial image to a display.

19. The computer readable storage medium of claim 18, wherein the first color scheme is a monochromatic color scheme.

20. The computer readable storage medium of claim 18, wherein the computer readable program instructions further cause the processor to:
apply an audible style to the first region.
